(19) United States
(12) Patent Application Publication
(10) Pub. No.: US 2016/0015627 A1
(43) Pub. Date: Jan. 21, 2016

(54) LACTOBACILLUS PLANTARUM NCC 2936 PREPARATIONS AND ORAL HEALTH

(71) Applicant: NESTEC S.A., Vevey (CH)

(72) Inventors: Michael John Callanan, Lausanne (CH); Edwin Ananta, Epalinges (CH); Stephane Duboux, St-Prex (CH); Anne Bruttin, Lausanne (CH); Ivana Jankovic, Epalinges (CH); Olga Sakwinska, Lausanne (CH); Gilles Bourdin, Fribourg (CH)

(21) Appl. No.: 14/774,354

(22) PCT Filed: Mar. 12, 2014
(86) PCT No.: PCT/EP2014/054801

§ 371 Date: Sep. 10, 2015

(30) Foreign Application Priority Data
Mar. 14, 2013 (EP) 13159170.3

(51) Int. Cl. A61K 8/99; A23K 1/00; A23L 1/30; A61Q 11/00; A61K 35/747

(52) U.S. Cl. CPC A61K 8/99; A61Q 11/00; A61K 35/747; A23L 1/3014; A23K 1/009; A23V 2002/00; A23Y 2220/67

(57) ABSTRACT

The present invention relates generally to compositions with a health benefit. In particular, the invention relates to bacterial preparations of Lactobacillus plantarum NCC 2936 (CNCM I-4026). An aspect of the invention is a composition for use in improving oral health comprising a bacterial preparation of Lactobacillus plantarum NCC 2936.

The present invention relates generally to compositions with a health benefit. In particular, the invention relates to bacterial preparations of Lactobacillus plantarum NCC 2936 (CNCM I-4026). An aspect of the invention is a composition for use in improving oral health comprising a bacterial preparation of Lactobacillus plantarum NCC 2936.

Antimicrobial agents have played a key role in improving public health in modern society. They find many diverse applications such as disinfecting surfaces, preserving food or as therapeutic agents in healthcare. One application of antimicrobial agents is in oral health, for example, antimicrobial mouth rinses can help prevent and reduce plaque and gingivitis. There are also an increasing number of elderly and medically compromised people who are potentially at risk for developing pneumonia due to aspiration of microorganisms in the oral cavity and throat. Patients with dysphagia are particularly at risk. Dysphagia is the medical condition of having difficulty in swallowing. Aspiration pneumonia often requires acute hospitalization and is life threatening among persons with dysphagia.

The risk of aspiration pneumonia is greatest when periodontal disease, dental caries and poor oral hygiene are compounded by swallowing disease, feeding problems and poor functional status. Aspiration pneumonia is thought to be caused by the aspiration of colonized nasopharynx or oropharynx material secondary to dysphagia. Forceful coughing, active ciliary transport and normal immune response are presumed to be protective but are inadequate. Microflora present in the oral cavity because of poor oral hygiene have been associated with aspiration pneumonia. WO2010/077795 discloses compositions to improve oral health comprising beneficial bacteria and also discloses a method for reducing the incidence of aspiration pneumonia by administering such compositions. However, there remains a need to develop alternative and/or complementary antimicrobial compositions, in particular compositions with wide ranging efficacy against harmful microorganisms and the ability to inhibit significant human pathogens such as Streptococcus pneumoniae, Pseudomonas aeruginosa and Haemophilus influenzae.

Lactic acid bacteria provide a promising source of antimicrobial activity for use in health care as they have a long and safe tradition in food fermentation processes. However, most lactic acid bacteria exhibit antimicrobial activity against only a narrow range of microorganisms. There is a need to provide antimicrobial agents from natural sources which show efficacy against a wide range of microorganisms, in particular, both Gram-positive and Gram-negative bacteria. For economies of scale during production it would be beneficial to provide antimicrobial agents which combine wide-ranging efficacy with a suitability for incorporation into a range of different materials. Such antimicrobial agents could be used in a variety of situations, for example in healthcare, or as food preservatives. There is also a persistent problem in that pathogenic bacteria over time develop resistance to existing antimicrobial compounds. There is therefore an ongoing need for the development of new antimicrobial compositions in order to continually meet the challenges presented by resistant strains.

An object of the present invention is to improve the state of the art and to provide alternative antimicrobial agents from a natural source which are effective against a wide spectrum of bacterial strains. In particular, it is an object of the invention to provide alternative compositions that can be used to improve oral health, especially compositions which have a further benefit of inhibiting respiratory illnesses associated with poor oral health, for example aspirational pneumonia caused or aggravated by poor oral health in dysphasia patients.

Any reference to prior art documents in this specification is not to be considered an admission that such prior art is widely known or forms part of the common general knowledge in the field. As used in this specification, the words “comprises”, “comprising”, and similar words, are not to be interpreted in an exclusive or exhaustive sense. In other words, they are intended to mean “including, but not limited to”. The object of the present invention is achieved by the subject matter of the independent claim. The dependent claims further develop the idea of the present invention.

The inventors were surprised to find that bacterial preparations of Lactobacillus plantarum NCC 2936 have wide-ranging antimicrobial properties. For example, the fermentation broth resulting after fermentation of Lactobacillus plantarum NCC 2936 was found to inhibit or delay the growth of a number of different microorganisms. The microorganisms delayed or inhibited by the broth included organisms associated with impaired oral health and associated respiratory illnesses such as Streptococcus pneumoniae, Streptococcus pseudopneumoniae, Streptococcus mitis, Streptococcus oralis, Staphylococcus aureus, Staphylococcus epidermidis, Haemophilus influenzae, Haemophilus parainfluenzae and Pseudomonas aeruginosa. The broth also was able to delay or inhibit other harmful organisms such as the food safety and food spoilage microorganisms Bacillus cereus, Bacillus licheniformis, Enterococcus faecium, Staphylococcus aureus, Listeria monocytogenes, Escherichia coli, Pseudomonas fluorescens, Salmonella enteriditis and Enterobacter cloacae. The fermentation broth resulting after fermentation of Lactobacillus plantarum NCC 2936 was found to, for example, inhibit or delay growth of both Gram-positive and Gram-negative bacteria. Although antimicrobial activity has been shown for other Lactobacillus plantarum strains, it surprising that a single Lactobacillus plantarum strain can inhibit such a wide range of bacteria. In addition, a Lactobacillus plantarum has not previously been identified which can inhibit Streptococcus pneumoniae or Haemophilus influenzae.

Bacterial preparations of Lactobacillus plantarum NCC 2936 have surprisingly been found to inhibit or delay the growth of organisms associated with impaired oral health and associated respiratory illnesses. Accordingly, an embodiment of the invention is a composition for use in improving oral health comprising a bacterial preparation of Lactobacillus plantarum NCC 2936. Oral health is essential to general health and quality of life. It is a state of being free from mouth and facial pain, oral and throat cancer, oral infection and sores, periodontal (gum) disease, tooth decay, tooth loss, and other diseases and disorders that limit an individual's capacity in biting, chewing, smiling, speaking, and psychosocial wellbeing.

Lactobacillus plantarum NCC 2936 is a sialic acid producing bacteria which previously was only known for its use in enriching the nutritional value of food by providing a source of sialic acid (EP2183984). Lactobacillus plantarum NCC 2936 was deposited with the Collection Nationale de Cultures de Microorganismes (CNCM), Institut Pasteur, 25 rue du Docteur Roux, F-75724 PARIS Cedex 15, France, on 25 Jun. 2008 and given the deposit number CNCM I-4026.

Consequently the present invention relates to a composition for use in improving oral health comprising a bacterial preparation of Lactobacillus plantarum NCC 2936 (CNCM I-4026). A bacterial preparation of Lactobacillus plantarum NCC 2936 has wide-ranging antimicrobial properties including the ability to inhibit or delay the growth of harmful bacteria found in the oral cavity. The inhibition of these bacteria improves oral health. Oral health is the health of the mouth and the first portion of the alimentary canal that receives food and saliva. Harmful bacteria may cause a number of different conditions of poor oral health, such as tooth decay, malodorous tongue flora and periodontal diseases such as gingivitis and periodontitis. The bacterial preparation of the composition for use in the present invention may comprise live Lactobacillus plantarum NCC 2936 bacteria. These live bacteria compete with harmful bacteria in the oral cavity, further improving oral health. The bacterial preparation of the present invention may comprise an aqueous suspension of bacteria, for example bacteria growing in a liquid nutritional medium.

The bacterial preparation may be a fermentation broth of Lactobacillus plantarum NCC 2936 or an extract thereof. The term fermentation broth refers to the culture medium resulting after fermentation of bacteria, including the bacteria and/or its component parts; unused raw substrates; and metabolites produced by the bacteria during fermentation. The fermentation broth may be a concentrated or dehydrated fermentation broth. Use of a fermentation broth is advantageous as the metabolites in the broth following fermentation with Lactobacillus plantarum NCC 2936 include a range of different materials with antimicrobial activities. For example the fermentation broth may contain low molecular weight organic acids, bacteriocins and antifungal peptides which have a beneficial antimicrobial effect. Preferably the fermentation broth is suitable for consumption by humans or pets, for example having all components appropriate for use in the preparation of food ingredients.

The bacterial preparation may be a fermentation broth of Lactobacillus plantarum NCC 2936 wherein the Lactobacillus plantarum NCC 2936 have been rendered non-viable, or an extract of a fermentation broth of Lactobacillus plantarum NCC 2936 wherein the Lactobacillus plantarum NCC 2936 have been rendered non-viable. The Lactobacillus plantarum NCC 2936 in the fermentation broth may have been rendered non-viable by any method known in the art. For example, the Lactobacillus plantarum NCC 2936 may be rendered non-viable by heat treatment, sonification, microwave irradiation, gamma irradiation, ultraviolet irradiation, mechanical cell disruption, chemical cell disruption or combinations thereof. Compositions which do not contain viable bacteria are easier to store and to introduce into products.

In the present invention, bacteria are described as non-viable when no viable cells and/or colony forming units can be detected by classical plating methods. Such classical plating methods are summarized in the microbiology book: James Monroe Jay, Martin J. Loessner, David A. Golden. 2005. Modern food microbiology. 7th edition, Springer Science, New York, N.Y. 790 p. Typically, the absence of viable cells can be shown as follows: no visible colony on agar plates or no increasing turbidity in liquid growth medium after inoculation and incubation under appropriate conditions (aerobic and/or anaerobic atmosphere for at least 24 h). In the bacterial preparation comprised within the composition of the present invention, at least 80%, preferably at least 90%, more preferably at least 95%, ideally at least 99.9% of the Lactobacillus plantarum NCC 2936 may be non-viable. The bacterial preparation may have a viable cell count of less than 1000 colony forming units (CFU) per ml, for example less than 200 CFU/ml, for further example less than 10 CFU/ml.

The bacterial preparation may be an extract of a fermentation broth of Lactobacillus plantarum NCC 2936. For example the Lactobacillus plantarum NCC 2936 may have been at least partially removed from the fermentation broth. Preferably the fermentation broth before the at least partial removal of the Lactobacillus plantarum NCC 2936 contains between 10⁵ and 10¹¹ colony forming units (CFU) per ml of Lactobacillus plantarum NCC 2936. In the case of Lactobacillus plantarum NCC 2936 which have been rendered non-viable, this term is to be understood as the amount of non-viable bacteria that is obtained from 10⁵ and 10¹¹ CFU/ml viable bacteria. In the present invention, “at least partially removed” means that at least 50% of the Lactobacillus plantarum NCC 2936 cells present in the bacterial preparation have been removed, for example at least 80% of the Lactobacillus plantarum NCC 2936 cells, or at least 90% of the Lactobacillus plantarum NCC 2936 cells, or at least 99.9%. For example all Lactobacillus plantarum NCC 2936 cells may have been removed. The cells may be at least partially removed by filtration and/or centrifugation.

The bacterial preparation may be the supernatant of a fermentation broth of Lactobacillus plantarum NCC 2936 after centrifugation. The supernatant of a fermentation broth of Lactobacillus plantarum NCC 2936 after centrifugation may have a cell count of less than 2000 CFU/ml. Such supernatants or filtrates may be concentrated or fractionated and may be dried to a powder. The bacterial preparation may be obtained as a by-product from the production of Lactobacillus plantarum NCC 2936, for example the production of Lactobacillus plantarum NCC 2936 for incorporation in a foodstuff as a source of sialic acid. Commonly in the production of bacteria, once the majority of bacterial cells have been removed or “harvested” from a fermentation broth, the rest of the broth is discarded. This is wasteful and it is often costly to ensure a safe and environmentally responsible disposal of the remaining broth. It is therefore an advantage that this by-product can be beneficially used in the current invention. The bacterial preparation may be obtained, for example obtainable, by a process comprising the steps of preparing a fermentation broth which contains between 10⁵ and 10¹¹ colony forming units per ml of Lactobacillus plantarum NCC 2936, removing substantially all the cells and concentrating the broth. The step of removing substantially all the cells may be performed by centrifugation and/or sterile filtration.

The improved oral health provided by the composition for use according to the invention may lead to a reduction in the incidence of aspiration pneumonia. Aspiration pneumonia is bronchopneumonia that develops due to the entrance of foreign materials into the bronchial tree, for example oral contents such as saliva, or nasal secretions. Depending on the acidity of the aspirate, a chemical pneumonitis may develop together with bacterial infection. The risk of aspiration pneumonia is greatest when periodontal disease, dental caries and poor oral hygiene are compounded by swallowing disease, feeding problems and poor functional status. Aspiration pneumonia is a common clinical consequence of dysphagia. Bacteria commonly implicated in aspiration pneumonia include Streptococcus pneumoniae, Staphylococcus aureus, Haemophilus influenzae, and Pseudomonas aeruginosa. It is therefore beneficial that a bacterial preparation of Lactobacillus plantarum NCC 2936 has been found to inhibit the growth of these bacteria.

The composition for use according to the invention may further comprise a thickener. Thickeners or thickening agents are substances which increase the viscosity of a solution or liquid/solid mixture without substantially modifying its other properties. Thickeners may be used to improve the suspension of other ingredients or emulsions which increases the stability of the product. Thickeners can also be used when medical conditions such as dysphagia cause individuals to have difficulty in swallowing. Increasing the viscosity of foods and drinks improves the ease and safety of swallowing. Thickened liquids play a vital role in reducing risk of aspiration for dysphagia patients. Thickeners are commonly based on polysaccharides (starches, vegetable gums, and pectin), or proteins. It is beneficial to be able to combine a composition for use in improving oral health according to the invention with a thickener, for example for use as a food and drink thickener for dysphagia patients. The increased viscosity not only improves the ease of swallowing, but also enhances the antimicrobial effect of the bacterial preparation of Lactobacillus plantarum NCC 2936 as the preparation remains in contact with surfaces within the oral cavity for longer.

The composition for use according to the invention may be administered to a dysphagia patient. In particular, the composition for use according to the invention may reduce the pathogenic bacterial load in the oropharynx and/or nasopharynx of a dysphagia patient. A reduction in the pathogenic bacterial load may be a reduction of at least one order of magnitude in the colony forming units of pathogenic bacteria, for example a reduction of at least two orders of magnitude. The pharynx in a human is the part of the throat situated immediately below the mouth and nasal cavity, and above the esophagus and larynx. The human pharynx is conventionally divided into three sections: the upper two being the nasopharynx and the oropharynx. Saliva and other material from the oral cavity may pass into the oropharynx and nasopharynx carrying with it bacteria, including harmful bacteria. The bacterial preparation of Lactobacillus plantarum NCC 2936 provides an unfavourable environment for the growth of pathogens. By reducing the levels of harmful bacteria in the oral cavity, the composition for use in the present invention reduces the levels of harmful bacteria in the oropharynx and nasopharynx. As an individual aspirates their saliva, the pathogenic bacterial load will be low and the likelihood of onset of aspiration pneumonia will be reduced. Processes such as coughing reintroduce material from the oropharynx and nasopharynx to the oral cavity so, in turn, a reduction in the levels of harmful bacteria in the oropharynx and nasopharynx is beneficial for oral health.

The composition for use according to the invention may reduce or prevent the incidence of dental caries. Dental caries are cavities in teeth caused by bacterial infections which demineralise and destroy hard dental tissues, usually by production of acid via bacterial fermentation of the food debris accumulated on the tooth surface. Today, caries remain one of the most common diseases throughout the world. The bacterial preparation of Lactobacillus plantarum NCC 2936 may be used to inhibit the bacteria responsible for dental caries and so improve oral health.

The composition for use according to the invention is not particularly limited in form. The composition may be selected from the group consisting of lozenges, lollipops, dissolvable films, mouthwashes, toothpaste, chewing gum, foodstuffs, drinks, nutritional formulas and pharmaceutical compositions. Lozenges are small medicinal tablets which are dissolved in the mouth. They provide a gradual release of their active ingredients to the oral cavity and throat. A bacterial preparation of Lactobacillus plantarum NCC 2936 may be comprised in a lozenge to provide an antimicrobial action. For example, a dry bacterial preparation of Lactobacillus plantarum NCC 2936 may be mixed with the other lozenge ingredients, or a liquid preparation may be comprised within a liquid centre, such as within a hollow sugar-shelled lozenge. Lollipops are boiled sweets formed on the end of a stick, generally round in shape. As for lozenges, lollipops may provide a slow release of active ingredients comprised within them as they dissolve. This is a pleasant and convenient way to deliver a composition for use in improving oral health.

Dissolvable films may conveniently be used to administer antimicrobial compositions in the mouth. They require very little water to dissolve and so may simply be allowed to dissolve in the patient's saliva. For example, a film may be prepared using hydrophilic polymers that rapidly dissolve on the tongue or buccal cavity together with a powdered bacterial preparation of Lactobacillus plantarum NCC 2936.

Mouthwashes or mouth rinses are therapeutic agents used to enhance oral hygiene. A mouthwash composition according to the invention may be used to inhibit harmful bacteria in the oral cavity, for example, bacteria responsible for cavities, gingivitis, and bad breath, or bacteria in the saliva of a dysphagia sufferer who is at risk of aspirating harmful bacteria in their saliva and so contracting aspiration pneumonia. Toothpastes may advantageously be used to apply a bacterial preparation of Lactobacillus plantarum NCC 2936 during daily tooth cleaning with a toothbrush. The mechanical action of the toothbrush, combined with abrasives in the toothpaste, removes bacteria harboured in dental plaque or in food trapped on the teeth, and this enhances the antimicrobial effectiveness of the bacterial preparation of Lactobacillus plantarum NCC 2936. The action of chewing, for example chewing a chewing gum, may increase saliva flow. The saliva produced carries with it calcium and phosphate which help strengthen tooth enamel. A bacterial preparation of Lactobacillus plantarum NCC 2936 may be comprised in chewing gum to provide an antimicrobial action in the mouth in addition to the beneficial ability of the chewing gum to mechanically stimulate saliva flow.

The composition for use according to the invention may be a foodstuff or a drink. The foodstuffs for use in the present invention are not particularly limited in form. For example the foodstuff may be confectionery products, fermented milks, fruit purées, food bars, compacted powders or non-compacted powders. The powders may be diluted in water, milk products or soya bean derivatives, or may be incorporated into other food. Drinks for use in the present invention may be for example fruit juices, fruit syrups, teas and herbal infusions.

The composition for use according to the invention may be a nutritional formula. This may, for example, be a complete nutritional formula which provides sufficient types and levels of macronutrients (protein, fats and carbohydrates) and micronutrients to be sufficient as a sole source of nutrition for the subject to which it is administered. The nutritional formula may also provide partial nutrition, to act as a supplement to the existing diet of the subject. Nutritional formulas provide the body with nutrients that it may urgently need when it is affected by poor oral health.

The composition for use in the invention may be a pharmaceutical composition, for example in the form of tablets, capsules or liquids such as sprays.

The composition for use according to the invention may be a food and drink thickener. Food and drink thickeners may be added to food or drinks which are to be consumed by dysphagia sufferers to make the food or drink easier to swallow. EP2493333 provides an example of a stable thickener formulation. It is convenient for dysphagia sufferers to have a single material to consume which both thickens their food or drink as well as providing an oral health benefit.

The composition for use according to the invention may be a pet care product or pet food. According to the American Veterinary Dental Society, more than 85 percent of dogs and cats show signs of oral disease by age four. Bacterial preparations of Lactobacillus plantarum NCC 2936 may be incorporated in or on a variety of pet care products to treat or prevent oral ill-health. For example a rubber dog chew may be coated with a dissolvable film containing a bacterial preparation of Lactobacillus plantarum NCC 2936. A powdered bacterial preparation of Lactobacillus plantarum NCC 2936 may be incorporated in dry dog or cat food.

The composition for use according to the invention may be a medical food. Medical foods are foods that are specially formulated and intended for the dietary management of a disease that has distinctive nutritional needs that cannot be met by normal diet alone. The composition for use according to the invention may be an oral nutritional supplement. Oral nutritional supplements are multi-nutrient liquid, semi-solid or powder products that provide macronutrients and micronutrients with the aim of increasing oral nutritional intake.

Those skilled in the art will understand that they can freely combine all features of the present invention disclosed herein. Where known equivalents exist to specific features, such equivalents are incorporated as if specifically referred to in this specification. Further advantages and features of the present invention are apparent from the non-limiting examples.

### EXAMPLES

### Example 1

### Lactobacillus plantarum NCC2936 Antimicrobial Activity

A vial of L. plantarum NCC 2936 was reactivated in 10 ml MRS broth (Difco) and incubated overnight at 30° C. in sterile anaerobic conditions (1st culture). A second culture was made by inoculating 10 ml of MRS broth with 2% of the previous culture (2nd culture). An inoculum was then prepared in 250 ml of MRS broth, inoculated at 2% with the 2nd culture. The final fermentation was performed in a 1 litre bioreactor containing MRS broth. Following the fermentation, the culture was centrifuged for 20 minutes at 3300×g (gravities) in sterile 1 L plastic bottles. The resulting pellet was discarded and the supernatant was filtered through a 0.2 μm filter. The filtered supernatant was frozen at −40° C. in a sealed aluminium bag and freeze-dried using a LyoBeta 35 instrument. The resulting powder was stored at 4° C. and rehydrated in the appropriate media for the inhibition assays.

Strains of typical microorganisms associated with food safety and spoilage and respiratory/oral cavities were collected from a variety of sources. In general, the selected strains were environmental isolates from food products and clinical sources. To perform the inhibition assays, cultures were grown overnight using appropriate media. Overnight cultures were diluted to achieve a starting inoculum of between 10³ and 10⁴ CFU/ml in normal growth media or media adjusted to pH 5.5. The freeze-dried filtered supernatant described above was rehydrated at 5% (w/v) in the growth media to form an antimicrobial stock solution. In standard multiwell plates, 20 μl of this stock antimicrobial solution was added to the diluted culture to give a final volume of 200 μl per well and a final concentration of 0.5% of the freeze dried material. The plate was loaded in a Bioscreen C Microbiological Analyser (Labsystems Helsinki, Finland) and incubated at 30° C. or 37° C. with shaking for 24-48 hours. The Optical Density (OD) was recorded every 15 minutes at 600 nm.

A delay or failure to record the Time To Detection (TTD) for each culture in the presence of the antimicrobial compared to the control was taken as the measure of inhibition. The TTD was determined when the OD₆₀₀ reading increased by 0.2 above the background for the media plus the antimicrobial. A delay was recorded when a slower TTD relative to the control was observed and this difference could vary considerably but generally required a minimum change of 2 hours. All assays were performed in triplicate. The results are presented in table 1 (effect on food safety and food spoilage microorganisms) and table 2 (effect on respiratory and oral microorganisms) below.

The results show that the bacterial preparation of Lactobacillus plantarum NCC 2936 has surprisingly wide-ranging antimicrobial properties; inhibiting or delaying the growth of microorganisms associated with impaired oral health and associated respiratory illnesses as well as food safety and food spoilage microorganisms. The bacterial preparation of Lactobacillus plantarum NCC 2936 is shown to inhibit or delay growth of both Gram-positive and Gram-negative bacteria. It has not previously been shown that a Lactobacillus plantarum can inhibit Streptococcus pneumoniae or Haemophilus influenzae.

TABLE 1
Inhibition assay results for NCC2936 super-
natant - Food safety/spoilage microorganisms.
Food safety/spoilage microorganism

| | |
|---|---|
| Gram Positive | Gram Negative |

| | | | |
|---|---|---|---|
| Bacillus cereus | I | Escherichia coli | I |
| Bacillus licheniformis | I | Pseudomonas fluorescens | I |
| Lactobacillus plantarum | X | Salmonella enteriditis | D |
| Enterococcus faecium | I | Enterobacter cloacae | D |
| Staphylococcus aureus | I | | |
| (food source) | | | |
| Listeria monocytogenes | I | | |

I = No TTD determined, X = Growth detected and D = Delay in TTD.

TABLE 2
Inhibition assay results for NCC2936 super-
natant - Respiratory/oral microorganisms.
Respiratory/oral microorganism

| Gram Positive | | Gram Negative | |
|---|---|---|---|
| Streptococcus pneumoniae | I | Haemophilus influenzae | I |
| Streptococcus pseudopneumoniae | I | Haemophilus parainfluenzae | I |
| Streptococcus mitis | I | Pseudomonas aeruginosa | I |
| Streptococcus oralis | I | | |
| Staphylococcus aureus | I | | |
| (clinical isolate) | | | |
| Staphylococus epidermidis | I | | |

I = No TTD determined, X = Growth detected and D = Delay in TTD.

### Example 2

### Mouthwash Comprising a Bacterial Preparation of Lactobacillus plantarum NCC2936

A sample of the freeze-dried supernatant of L. plantarum NCC 2936 from Example 1 is used to formulate a mouthwash with the following composition:

12% ethanol
6% glycerine
0.5% free-dried Lactobacillus plantarum NCC2936 supernatent

### 0.2% PEG 40 Hydrogenated Castor Oil (Cremophor RH40)

0.2% flavour (e.g. peppermint)
0.05% saccharin
the balance being water.

1. A method for improving oral health comprising administering to an individual in need of same a bacterial preparation comprising Lactobacillus plantarum NCC 2936.

2. Method according to claim 1 wherein the bacterial preparation is a fermentation broth of Lactobacillus plantarum NCC 2936 or an extract thereof.

3. Method according to claim 2 wherein the Lactobacillus plantarum NCC 2936 have been rendered non-viable.

4. Method according to claim 2 wherein the Lactobacillus plantarum NCC 2936 have been at least partially removed from the fermentation broth.

5. Method according to claim 1 wherein the bacterial preparation is the supernatant of a fermentation broth of Lactobacillus plantarum NCC 2936 after centrifugation.

6. Method according to claim 1 wherein the improved oral health leads to a reduction in the incidence of aspiration pneumonia.

7. Method according to claim 1 wherein the preparation further comprises a thickener.

8. Method according to claim 1 wherein the preparation is administered to a dysphagia patient.

9. Method according to claim 1 wherein the improvement in oral health is a reduction or prevention of the incidence of dental caries.

10. Method according to claim 1 wherein the preparation is selected from the group consisting of lozenges, lollipops, dissolvable films, mouthwashes, toothpaste, chewing gum, foodstuffs, drinks, nutritional formulas and pharmaceutical compositions.

11. Method according to claim 1 wherein the preparation is a food and drink thickener.

12. Method according to claim 1 wherein the preparation is a pet care product or pet food.

13. Method according to claim 1 wherein the preparation is a medical food.

14. Method according to claim 1 wherein the preparation is an oral nutritional supplement.
